(19) United States
(12) Patent Application Publication
(10) Pub. No.: US 2026/0021150 A1
(43) Pub. Date: Jan. 22, 2026

(54) COMBINATION THERAPY OF TRADITIONAL CHINESE AND MODERN MEDICINE FOR TREATMENT OF PATIENTS WITH AMYOTROPHIC LATERAL SCLEROSIS

(71) Applicant: Integrated Molecular Medicine Limited, Hong Kong (HK)

(72) Inventors: Edward S. YANG, Hong Kong (HK); Jian-Bo LIU, Taiyuan Cit (CN); Xiao-Yue CHANG, Hong Kong (HK)

(21) Appl. No.: 18/778,078

(22) Filed: Jul. 19, 2024

(51) Int. Cl. A61K 36/185; A61K 31/4152; A61K 31/428; A61K 31/711; A61P 25/28

(52) U.S. Cl. CPC A61K 36/185; A61K 31/4152; A61K 31/428; A61K 31/711; A61P 25/28

(57) ABSTRACT

Disclosed is a method for diminishing effects of Amyotrophic Lateral Sclerosis (ALS) in administering to the predisposed subject effective amounts of Traditional Chinese Medicine (TCM) or in a combination of a Western medicine, wherein the TCM including: (1) Ge Gen, (2) Dang Gui, (3) Dan Shen, (4) Dang Shen, (5) Huang Qi, (6) Zi Su Zi, (7) Da Zao, (8) Chai Hu, (9) Huang Qin, (10) Hong Hua, (I I) Yu Jin, (12) Da Huang, (13) Hua Jiao, (I4) Gan Cao, (15) Mai Dong, (I6) Wu Wei Zi, (17) Fu Zi, (18) Ren Shen, (I9) Fu Ling, (20) Shi Gao, (21) Mu Li, (22) Gui Zhi, and (23) Che Qian Zi; or, extracts thereof in amounts equivalent to the amounts of the raw materials of the group of ingredients. The patent is targeted on the clinical efficacy of the different pathological forms of ALS disease manifestation.

### CROSS-REFERENCE TO RELATED APPLICATIONS

Not applicable.

### BACKGROUND OF THE INVENTION

The present invention relates to a combination therapy making use of Traditional Chinese Medicine with or without Modern Western Medicine for treatment of patients with Amyotrophic Lateral Sclerosis (ALS).

Neurodegeneration refers to the progressive and irreversible loss of structure or function of neurons, which are the specialized cells that transmit nerve impulses in the brain and spinal cord. Amyotrophic lateral sclerosis (ALS) is recognized as a neurodegenerative disease caused by the loss of motor neuron. It's lack of effective therapies is hampered by an imperfect understanding of the biological processes that trigger ALS and promote disease progression. At early stage, it mainly manifests as muscle atrophy and weakness in the limbs. At later stages, it may be combined with bulbar paralysis to cause dysarthria, dysphagia, and dyspnea. Neurological examination shows active or hyperactive tendon reflexes. Electromyography often reports as neurogenic damage. While about 5% of the patients received their defected genes through the familial ALS (fALS), about 95% are sporadic (sALS) having no known family members with the same disorder. At the present, ALS is considered as the most challenging disease in modern medicine, lacking a definite understanding of the mechanism of its pathogenesis.

The disease was first reported by Charles Bell (1824) but the clinical and pathological description is credited to Jean-Martin Charcot (1874) who coined the term amyotrophic lateral sclerosis for spinal-cord hardening caused by un-nourished muscle. It is named after Lou Gehrig in the US, a great baseball player (1939) and publicly identified with Stephen Hawking (1963), a distinguished English physicist. People who are diagnosed with amyotrophic lateral sclerosis today face the same devastating news since Bell's time, that there is no effective treatment even with the advance of molecular medicine, science, and technology. Among the neurodegenerative disorders, ALS is the most fatal disease in rapidity, with death in 2-3 years after diagnosis. Pathophysiology is better understood after 3 decades of intense research, yet with no effective treatment. At present, we have three Food and Drug Administration (FDA) approved drugs, but serious toxic byproducts and cell damages are produced by these drugs with none for healing. Worst, the disease shows characteristically heterogeneous without proven biomarkers in clinical trials, making a one-size fits all solution unworkable and unmeasurable. For this reason, multidrug trials are needed to address different functions, mechanisms, or disabilities of the disease stretching out for many years, and regrettably, no one managed to overcome and survive after 200 years. The present disease model has been developed in the last few months.

To track down the trace of the origin of ALS, it starts with the symptoms of upper and lower motor neuron abnormalities in the brain, spinal and CNS. The most obvious disorders involve speech and swallowing difficulties in bulbar onset or arm and leg disabilities and followed by limp onset and progressive paralysis. Other symptoms are weight loss, cramps, respiratory failures and fasciculations. The altered function of upper neurons leads to spasticity and brisk deep reflexes and symptoms of lower motor neuron abnormalities including muscle wasting and weakness. Given that there are no specific biomarkers of ALS, the diagnosis is established based on clinical features as ratified by consensus criteria.

Mutation studies include SOD1, C9orf72, and TDP-439331K human and mice producing phenotypes pathogenic ALS (Kienan 2021). Many of these mutations affect proteins that are involved in gene expression and regulation via the regulation of transcription, microRNA processing, RNA maturation and splicing. The mutations linked to ALS are commonly grouped according to whether they alter proteostasis, RNA function or the cytoskeleton. But, in all honesty, high-quality scientific papers and clinical trials have delivered an unusual Manhattan-style traffic jam and frustration. With billions of research funding, millions of patients, tens of thousands of physicians, the best genetic and molecular research tools, there has been no ALS cure in sight.

Some of these examples are discussed in the following prior arts.

U.S. Pat. No. 9,289,459B2 discloses the use of a composition for promoting neuronal growth of neurons in tissues of the central or peripheral nervous system. The prior art also discloses a method for inducing proliferation or differentiation of neuronal cells. However, the prior art may not adequately address degenerative diseases, particularly amyotrophic lateral sclerosis (ALS). Additionally, the prior art primarily targets repairing cell damage and facilitating muscle healing, rather than specifically addressing the proliferation or differentiation of neuronal cells. This approach may pose limitations in effectively managing degenerative diseases. The prior art is primarily geared towards promoting the growth, proliferation, and differentiation of neuronal cells to support the development of healthy cells exclusively. Consequently, its effectiveness may be constrained when addressing injured or mutated cells, as well as those in a diseased state. Additionally, the prior art may not adequately address the complexities associated with astrocytes, microglia, and neurons, nor the specific challenges posed by diseases with separate limb and bulbar onsets. Furthermore, it may not adequately manage the adverse side effects resulting from genetic, molecular, and pharmacological injuries, nor effectively navigate the interactions between healthy and damaged molecules and organs.

China Patent No. 108785586B discloses a traditional Chinese medicine composition for adjuvant therapy of motor neuron diseases and its application. The Chinese medicinal composition comprises radix Puerariae, rhizoma Drynariae, Achyranthis radix, fructus Alpinae Oxyphyllae, semen Platycladi, Poria, rhizoma Ligustici Chuanxiong, radix Angelicae sinensis, fructus Schisandrae chinensis, radix scrophulariae, Saviae Miltiorrhizae radix, radix Codonopsis, flos Lonicerae, radix Ophiopogonis, radix Platycodi, cortex et radix Polygalae, radix rehmanniae Preparata, bupleuri radix, radix Saposhnikoviae, radix astragali, Scutellariae radix, Coptidis rhizoma, fructus Aurantii, radix Paeoniae Rubra, and Glycyrrhrizae radix. The prior invention utilizes the multi-component, multi-path and multi-target synergistic effect of the traditional Chinese medicine to achieve the auxiliary treatment of the motor neuron diseases and has better effect on the torsion of the disease development. However, the prior art may not provide a comprehensive solution for treating degenerative diseases, especially ALS. While the prior invention is specifically tailored for ALS, the treatments it offers may not adequately address diseases with intricate progression patterns and diverse manifestations. Additionally, the prior art may not adequately address the complexities associated with astrocytes, microglia, and neurons, nor the specific challenges posed by diseases with separate limb and bulbar onsets. Furthermore, it may not adequately manage the adverse side effects resulting from genetic, molecular, and pharmacological injuries, nor effectively navigate the interactions between healthy and damaged molecules and organs.

China Patent No. 104825525A discloses a traditional Chinese medicine composition for treating amyotrophic lateral sclerosis and a preparation method thereof. The traditional Chinese medicine composition is mainly prepared from the following raw materials in parts by weight: 2-5 parts of epimedium, 1-3 parts of atractylodes macrocephala stir-fried with bran and 1-3 parts of Poria cocos. Besides, the prior art further discloses the preparation method of the traditional Chinese medicine composition. Pharmacological research proves that the traditional Chinese medicine composition provided by the invention has the effects of improving limb strength, delaying the muscle atrophy process, prolonging lifetime, and delaying the apoptosis of motor neuron. Although the prior art constructed for ALS treatments, but it is not the prescriptions invented to treat diseases with complex developments and manifestations. Thus, the prior art may not provide a comprehensive solution for treating degenerative diseases, especially ALS. Besides, the prior art may not adequately address the complexities associated with astrocytes, microglia, and neurons, nor the specific challenges posed by diseases with separate limb and bulbar onsets. Furthermore, it may not adequately manage the adverse side effects resulting from genetic, molecular, and pharmacological injuries, nor effectively navigate the interactions between healthy and damaged molecules and organs.

U.S. Pat. No. 9,393,290B2 discloses compositions and methods for treating stroke patients using TCM and a Western medicament used for the treatment of stroke patients. The prior art focuses on the treatment for stroke patients, although such treatment may be applicable for ALS patients. Hence, it may be less effective for ALS treatment in particular. The prior art may not adequately address the complexities associated with astrocytes, microglia, and neurons, nor the specific challenges posed by diseases with separate limb and bulbar onsets. Furthermore, it may not adequately manage the adverse side effects resulting from genetic, molecular, and pharmacological injuries, nor effectively navigate the interactions between healthy and damaged molecules and organs.

Despite having various inventions using traditional Chinese medicine, with or without combination with existing therapies, there remains a necessity to offer a more comprehensive treatment strategy for degenerative diseases. This approach should not only target the disease itself but also prioritize patient protection.

### SUMMARY OF THE INVENTION

Therefore, it is an objective of the present invention to provide a cocktail of drug with healing capacities and low-to-zero adverse effects in treating Amyotrophic Lateral Sclerosis (ALS) by targeting on the pathogenesis of neuromyopathy and the syndrome differentiation of neuromyopathy in patients with ALS.

It is also an objective of the present invention to provide a cocktail of drug with workable and measurable outcomes in combatting the heterogenous characteristics of ALS.

Another objective of the present invention to provide a cocktail of that can be used a single treatment for ALS or in combination with an agent of Western medicine for the treatment of ALS.

Generally, the present invention relates to a method and products for diminishing effects of ALS or neurodegeneration in a predisposed subject comprising administering to the predisposed subject effective amounts of Traditional Chinese Medicine (TCM) selected from a group of ingredients comprising: (1) Ge Gen (Radix puerariae, Lobed Kudzuvine Root), (2) Dang Gui (Radix angelicae, Chinese Angelica or Female Ginseng), (3) Dan Shen (Salviae miltiorrhizae, Red sage), (4) Dang Shen (Codonopsis radix, Asiabell Root), (5) Huang Qi (Radix astragali, Milkvetch Root), (6) Zi Su Zi (Perillae fructus, Purple Perilla Fruit), (7) Da Zao (Jujubae fructus, Jujube), (8) Chai Hu (Bupleuri radix, Chinese Thorawax Root) (9) Huang Qin (Scutellariae radix, Baikal Skullcap Root), (10) Hong Hua (Carthami flos, Safflower), (111) Yu Jin (Curcumae radix, Turmeric), (12) Da Huang (Rhei radix, Rhubarb), (13) Hua Jiao (Zanthoxyli pericarpium, Pricklyath Pericarp), (14) Gan Cao (Glycyrrhizae radix, Liquorice), (15) Mai Dong (Ophiopogonis radix, Dwarf Lilyturf Root), (16) Wu Wei 2i (Schisandrae chinensis fructus, Chinese Magnoliavine Fruit), (17) Fu Zi (Aconiti lateralis, Monkshood Daughter Root), (18) Ren Shen (Ginseng radix, Ginseng), (19) Fu Ling (Poria cocos, Buead Tuckahoe,) (20) Shi Gao (Gypsum fibrosum, Gypsum), (21) Mu Li (Ostreae concha, Oyster shell), (22) Gui Zhi (Cinnamomi ramulus, Cassiabarktree Twig), and (23) Che Qian Zi (Plantaginis semen, Asiatic Plantain Seed); or, extracts thereof in amounts equivalent to the amounts of the raw materials of the group of ingredients.

### BRIEF DESCRIPTION OF DRAWINGS

The features of the invention will be more readily understood and appreciated from the following detailed description when read in conjunction with the accompanying drawings of the preferred embodiment of the present invention, in which:

FIG. 1 illustrates the age distribution of patients.

FIG. 2 shows the effectiveness on limb and bulbar onsets along with prescribed medication in percentage in the present invention.

FIG. 3 illustrates the data from the control unit, comparing patients treated with Riluzole alone to those receiving Riluzole in conjunction with Chinese herbal medicine.

FIG. 4 depicts the survival outcomes observed in the animal trial, comparing the use of herbal medicine with the untreated control group.

FIG. 5 illustrates the weight fluctuations observed in 24 SOD1 transgenic mice over an 11-week period, comparing those administered with Chinese herbal medicines without Riluzole to those left untreated.

### DETAILED DESCRIPTION OF THE INVENTION

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

Generally, the present invention discloses a method for diminishing effects of Amyotrophic Lateral Sclerosis (ALS) or neurodegeneration in a predisposed subject comprising administering to the predisposed subject effective amounts of Traditional Chinese Medicine (TCM) selected from a group of ingredients comprising: (1) Ge Gen (Radix puerariae, Lobed Kudzuvine Root), (2) Dang Gui (Radix angelicae, Chinese Angelica or Female Ginseng), (3) Dan Shen (Salviae miltiorrhizae, Red sage), (4) Dang Shen (Codonopsis radix, Asiabell Root), (5) Huang Qi (Radix astragali, Milkvetch Root), (6) Zi Su Zi (Perillae fructus, Purple Perilla Fruit), (7) Da Zao (Jujubae fructus, Jujube), (8) Chai Hu (Bupleuri radix, Chinese Thorawax Root) (9) Huang Qin (Scutellariae radix, Baikal Skullcap Root), (10) Hong Hua (Carthami flos, Safflower), (11) Yu Jin (Curcumae radix, Turmeric), (12) Da Huang (Rhei radix, Rhubarb), (13) Hua Jiao (Zanthoxyli Pericarpium, Pricklyath Pericarp), (14) Gan Cao (Glycyrrhizae radix, Liquorice), (15) Mai Dong (Ophiopogonis radix, Dwarf Lilyturf Root), (16) Wu Wei 2i (Schisandrae chinensis fructus, Chinese Magnoliavine Fruit), (17) Fu Zi (Aconiti lateralis, Monkshood Daughter Root), (18) Ren Shen (Ginseng radix, Ginseng), (19) Fu Ling (Poria cocos, Buead Tuckahoe,) (20) Shi Gao (Gypsum fibrosum, Gypsum), (21) Mu Li (Ostreae concha, Oyster shell), (22) Gui Zhi (Cinnamomi ramulus, Cassiabarktree Twig), and (23) Che Qian Zi (Plantaginis semen, Asiatic Plantain Seed); or, extracts thereof in amounts equivalent to the amounts of the raw materials of the group of ingredients.

In accordance with a preferred embodiment of the present invention, the effect of neurodegeneration in the predisposed subject is due to Amyotrophic Lateral Sclerosis (ALS).

The present invention provides a TCM for treating motor neuron diseases, specifically for Amyotrophic Lateral Sclerosis (ALS). The purpose of formulating the formula is to adjust the functions of internal organs. Therefore, in addition to the organic combination of the functions of the medicines, as well as to complement each other and reflect their overall effects, the medicine is combining the taste of sour and sweet to enhance yin, opening the taste of acrid while lowering level of bitterness, combining the taste of sweet cold and bitter cold, as well as sweet warm and sweet cold, realizing the goal of regulating internal organs, and treating both symptoms and root causes. The use of Chai Hu (Bupleuri radix, Chinese Thorawax Root), Huang Qin (Scutellariae radix, Baikal Skullcap Root), Zi Su Zi (Perillae fructus, Purple Perilla Fruit), Dang Shen (Codonopsis radix, Asiabell Root), Hua Jiao (Zanthoxyli pericarpium, Pricklyash Pericarp), Gan Cao (Glycyrrhizae radix, Licorice), and Da Zao (Jujubae fructus, Jujube), which are cool in nature, is to form a harmonious basic formula, which play a role in coordinating the whole ingredients. The prescribed medicines are excellent in defence and superbly protective, maintaining the homeostasis in an integrated system to annihilate the mutated without injuring the healthy cells and proteins. The supplements of Huang Qi (Radix astragali, Milkvetch Root), Ge Gen (Radix puerariae, Lobed Kudzuvine Root), Dan Shen (Salviae miltiorrhizae, Red Sage), Yu Jin (Curcumae radix, Turmeric), Dang Gui (Radix angelicae, Chinese Angelica or Female Ginseng), Hong Hua (Carthami flos, Safflower), Da Huang (Rhei radix, Rhubarb), Mai Dong (Ophiopogonis radix, Dwarf Lilyturf Root), Wu Wei Zi (Schisandrae chinensis fructus, Chinese Magnoliavine Fruit), Fu Zi (Aconiti lateralis, Monkshood Daughter Root), Ren Shen (Ginseng Radix, Ginseng), Fu Ling (Poria cocos, Bread Tuckahoe), Shi Gao (Gypsum fibrosum, Gypsum), Mu Li (Ostreae concha, Oyster Shell), Gui Zhi (Cinnamomi ramulus, Cassiabarktree Twig), and Che Qian Zi (Plantaginis semen, Asiatic Plantain Seed), are to help strengthen organs of spleen and stomach while resulting in a better liver and kidney, replenishes qi while activating blood flow; and dredges meridians and collaterals, and at the same time treating both the symptoms and the root causes.

The present invention employs the principle of whole-body coordination and targeting the local part in the predisposed subject. From its sites of symptoms in a patient as observed by a trained physician, ALS may be considered as a localized motor neuron disease identifiable in limp and bulbar manifestations. In clinical practice of Chinese medicine, localized diseases that exhibit pathological changes may not be clearly shown in the whole body but appear as localized functional disabilities. The pathological symptoms with chronic progression are relatively stable to allow certainty of prescription used in their treatment. When a localized lesion appears, various therapies can be used as treatment. In modern medicine, surgical oncology is an example of localized treatment for localized diseases. Sometimes, localized diseases do not manifest as localized symptoms, but exhibit obvious deficiency or excess patterns in the whole body. In such cases, it may be given medicine for a whole-body treatment.

In some diseases, while a local area is infected, the normal function of the whole body is affected, causing an overall imbalance. In turn, this imbalance gives rise to suffering of larger areas making it necessary to treat both the local area and the whole body as a unit. Each local area must obey the overall treatment plan, and only through overall coordination can local improvement be achieved. While each local area must obey the overall improvement plan, only through overall coordination can local improvement be achieved. Whole-body treatment uses a whole-body coordinated prescription in combination with a local prescription to treat the patient. In Chinese medicine, many of the coordinating prescriptions used in clinical practice are based on the theory of local and whole-body dualism, which emphasizes the coordination of the whole body while highlighting local treatment. In opposition, modern western medicine focuses on targeted local therapy.

Local diseases are often persistent and treating them requires stability in the prescription and medication. Local diseases rarely spread from one area to another, so the prescription and medication for local treatment should remain consistent. This means that the prescription shall be adhered to continually. The development process of a local disease has an essential element that determines the course of the disease. Until the disease has run its course, it will not be cured. The treatment must be based on the disease, and the prescription must not be changed if the essence of the disease has not been changed; otherwise, it will be ineffective. The persistence of local diseases requires a certain approach to treatment. After diagnosis, if a prescription does not work, it should not be changed randomly. Instead, the root cause of the disease must be addressed. If the diagnosis is incorrect, the prescription shall be corrected to address the underlying problem, not to treat the symptoms. The correctness of the prescription is essential to the success of the treatment. The effectiveness of the treatment can only be realized by understanding the essence of the disease and adhering to the consistent treatment. Studying the rules of disease occurrence and development is essential to finding a suitable treatment. The rules that are discovered must be able to be repeated and applied in clinical practice.

As such, one of the principles of treating a local disease is to treat both the local and overall body conditions. The treatment of the local condition is also a form of adjustment, through which the local and overall conditions can be organically unified, maintaining a dynamic balance and achieving the purpose of treatment. In Chinese herbal medicine treatment, it is recognized only through coordination of the whole body can improve local condition, and the prescription for coordinating the whole body is Xiao Chai Hu Tang, XCHT (Minor Bupleurum Decoction). XCHT is the first prescription for treating systemic symptoms and it is also the preferred prescription for coordinating local conditions. XCHT is an optimal coordinating prescription, and on this basis, local treatment can be added by selecting appropriate herbal formulas or medications. Focusing on the local aspect is an organic combination of universal and specific treatments, reflecting the principle of coordinating the whole body while targeting the local part. The local treatment reflects the antagonism between the body and the pathogens, and a separate targeted treatment is needed. Effectively, the prescriptions are based on a principle of dualism, treating the local and the whole body.

In accordance with a preferred embodiment of the present invention, the TCM is in a form of decoctions, granules, capsules, powders, water pills, tablets, mixtures, oral liquids or other dosage forms. The Chinese medicines of this present invention are processed according to conventional methods.

In accordance with a preferred embodiment of the present invention, the TCM is administered to the predisposed subject for a period of at least 3 months consecutively for effective results.

For example, for the administration of medicine orally, the medicine may be administered to the predisposed subject 6 capsules each time, 3 times a day with warm water as least 30 minutes before a meal. The treatment period of 3 months is adequate for patients with mild symptoms. The course of treatment of the present invention generally to be taken for 180 days. However, it can be taken for a prolonged period, i.e. for years. For those with more severe symptoms, an increased dosage can be used. The acute toxicity test of this invention shows that there is no acute toxic reaction, proving its safety.

In accordance with a preferred embodiment of the present invention, the TCM is administered on its own or in a combination with an agent used in Western medicine with a glutamate inhibitor namely Riluzole, Edaravone, or Toferson, or in other similar Western medicine.

In Chinese medicine, coordination between various herbal components is relatively easily performed. But in contrast with modern Western medicine, the whole spectrum of herbs has no suitable isolated source that can be used as a component of focused energy for local healing. On the other hand, western medicine in treating ALS is a single targeted event. As such, the present invention uses modern western drug in the collaboration. In other words, Chinese herbal medicine is combined with the modern Western medicine in the treatment of patients with ALS, and motor neuron diseases.

Adverse effects caused by Riluzole is considered minor by most physicians. But the use of Riluzole may include weakness, drowsiness, dizziness, diarrhoea, nausea, vomiting, headache, lung injury, hepatoxicity, as well as stomach and muscle pain, loss of appetite or sleep. These side effects are good tests of the herbal medicine as biomarkers for its ability to minimize the potential injuries.

The use of Chinese medicine helps as anti-side effect of the Western medicine. The TCM acts as a matching pair to neutralize or compensate for the offensive toxicity of Riluzole, the modern western drug. It is well known that TCM has few adverse side-effects as compared to western medicines. As an example, XCHT (Bupleurum chinense) is found to be analgesic, immune-regulated, anti-bacterial, anti-convulsant, anti-depression, anti-diabetic, anti-epileptic, anti-microbial, anti-pyretic, anti-virus, and anti-inflammation. It potentially brings back normalcy by delivering therapeutic functions by minimizing or eliminating these negative side-effects of Western medicine.

Further, in treating a disease, there are two logical approaches. One focuses on the offensive, eliminating all viruses, defects, and mutants in the body, whilst another one focuses on the defense, strengthening the cells, tissues, and organs to become normal with healthy immunity, not regarding time and money being the unmentioned third party in the treatment selection.

In this case, the modern Western medicine delivers a torch-earth targeted attack, killing all the bad cells along with the good ones, hardly any discrimination or inhibition. Science and technology developed by modern laboratories able to provide the convenience of pills, or injections. Whilst, Chinese medicine, which created over two millennia, does not have the luxury of a speedy resolution, although it is able to be used as the slow protective method of reconditioning, regulating, and coordinating the whole body to repair, rectify, clean up the damages, restore the function, and maintain the body's balance and equilibrium. The word harmony or homeostasis is a condition preferred in TCM. It allows time to remove the toxicity, localize or separate the diseased cells or organs, a significant departure from the current chemical based pharmaceutical practice. Side effects are clearly implicated by the treatment methods one employs.

The utilization of XCHT (Bupleurum chinense) helps to coordinate, preserve, nurture, protect, regulate, bringing order from disorders, construction from destruction, a genuine but slow process with multi-purposes and dimensions. It transforms the medicine into the defender for the body and neutralizes the offending species against disorder and disruption. It does not function simply as anti-cancer, anti-inflammation, anti-depression, and all the negative of the disease. It brings positive energy and minimizes pain, as a working healthy organism back to purpose, function, and normalcy. Most important, it takes more time to establish an environment for the organic body and medicine to work together as a team, to achieve a sense of balance, equilibrium, and for survival itself, the physical and physiological unity of overcoming the infirmities and injuries.

In general, the slow response is recognized as a drawback. As such, TCM which is defensive in nature requires a matching offensive medical component. With Western medicine like Riluzole, it allows the modern drug to kill the pathogens or mutated cells in the diseased tissues while enabling the TCM to eliminate the side-effects by breaking up the toxic byproducts, avoiding cell damage and protecting the healthy cells. The combined medicine is intended to treat both disease and side effects at the same time with minimum destruction and injury to the patient.

It is important to note that polysaccharides are natural antigens that may trigger an immune response in Chai Hu and other elements, which are key herbal components in the proposed ALS formula. Immune function is critical for homeostasis of organs throughout the whole body. Making use of the formulation in the present invention to stop the formation of or block neurotoxin as a promising approach to treat chronic neurological diseases and acute CNS injuries.

A patient has both malignant and healthy cells sharing neighboring space in sub-microns. Cell division or mutation is expected to be the mechanism of the spreading of the disease. The present invention proposes two kinds of medication, the targeted western chemical attacking the diseased molecules and the Chinese herbal prescriptions with chemicals as a nutrient and defensive element for the heathy tissues and organs. The two drugs will not go against each other as one is attacking the malignancy and the other to minimize the side-effects, i.e., both are for healing of the body. The actual separation may come from the signals themselves. Drugs are likely to transport through the digestive system, lymphatic paths, or the blood vessels. As a result, the discrimination likely comes at the cell or molecular nutrient supplies and contact interface. The more different in physical and chemical attributes between the western drugs and the TCM, the better to allow the separation of offensive and defensive functions. Realtime observation does not seem realistic even if it is possible. MRI or NMR might be helpful as a method of observation at the end and not time in between. Nevertheless, with different functions involved, it appeals to the body's adaptation.

To build up a panacea of the integrated East and West health preserver, the present invention chooses TCM based on XCHT or Bupleurum chinense with some variations. Limited clinical evidence for Amyotrophic Lateral Sclerosis is available focusing on sporadic ALS, the truly incurable disease without an effective FDA approved name brand.

In accordance with a preferred embodiment of the present invention, the agent used in the Western medicine is provided for simultaneous, separate or sequential use in treatment of the predisposed subject.

In accordance with a preferred embodiment of the present invention, the use of the traditional Chinese medicine (TCM) components to manufacture the medicament of the treatment for the neurodegenerative disease including but not limited to repairing cell damages and muscle healing.

The present invention further asserts a product comprising a Chinese medicament comprising of 10-15 units of Huang Qi (Radix astragali, Milkvetch Root), 5-10 units of Ge Gen (Radix puerariae, Lobed Kudzuvine Root), 3-6 units of Dang Gui (Radix angelicae, Chinese Angelica or Female Ginseng), 1.5-3 units of Hong Hua (Carthami flos, Safflower), 3-6 units of Dan Shen (Salviae miltiorrhizae, Red Sage), 1.5-3 units of Yu Jin (Curcumae radix, Turmeric), 1-2 units of Da Huang (Rhei radix, Rhubarb), 1.5-3 units of Chai Hu (Bupleuri radix, Chinese Thorawax Root), 1.5-3 units of Huang Qin (Scutellariae radix, Baikal Skullcap Root), 3-6 units of Zi Su Zi (Perillae fructus, Purple Perilla Fruit), 3-6 units of Dang Shen (Codonopsis radix, Asiabell Root), 1-2 units of Huajiao (Zanthoxyli pericarpium, Pricklyash Pericarp), 1-2 units of Gan Cao (Glycyrrhizae radix, Licorice), and, 1.5-3 units of Da Zao (Jujubae fructus, Jujube). The product is known as Ji Fu Ling.

In accordance with a preferred embodiment of the present invention, the present invention further discloses a product comprising a Chinese medicament comprising of 1-2 units of Ren Shen (Ginseng radix, Ginseng), 1.5-3 units of Fu Zi (Aconiti lateralis, Monkshood Daughter Root), 1.5-3 units of Fu Ling (Poria cocos, Bread Tuckahoe), 3-6 units of Mai Dong (Ophiopogonis radix, Dwarf Lilyturf Root), 1.5-3 units of Wu Wei Zi (Schisandrae Chinensis Fructus, Chinese Magnoliavine Fruit), and, 6-10 units of Huang Qi (Radix astragali, Milkvetch Root) The product is known as Yuan Qi Tang.

In accordance with a preferred embodiment of the present invention, the present invention further discloses a product comprising a Chinese medicament comprising of 3-6 units of Shi Gao (Gypsum fibrosum, Gypsum), 1-2 units of Gui Zhi (Cinnamomi ramulus, Cassiabarktree Twig), 3-6 units of Mu Li (Ostreae concha, Oyster Shell), 1-2 units of Che Qian Zi (Plantaginis semen, Asiatic Plantain Seed), 1.5-3 units of Chai Hu (Bupleuri radix, Chinese Thorawax Root), 1.5-3 units of Huang Qin (Scutellariae radix, Baikal Skullcap Root), 3-6 units of Zi Su Zi (Perillae fructus, Purple Perilla Fruit), 3-6 units of Dang Shen (Codonopsis radix, Asiabell Root), 1-2 units of Hua Jiao (Zanthoxyli pericarpium, Pricklyash Pericarp), 1-2 units of Gan Cao (Glycyrrhizae radix, Licorice), and, 1.5-3 units of Da Zao (Jujubae fructus, Jujube). The product is known as An Shen Tang.

The preparation method of the traditional comprising the steps of: preparing a total of 10 kg of the TCM components based on the formulations; decocting the TCM components for each formulation two times for 2 hours and 1.5 hours respectively; and, combining and filtering the decocted TCM components forming concentrated TCM components; drying the concentrated TCM components at a pre-determined temperature around 80C for a period of 4-8 hours. crushing the dried concentrated TCM components into fine powder; and, encapsulating the fine powder into capsules for consumption. This process has the potential to produce approximately 2000 capsules in a single batch. Further, the medicine is advised to be taken half an hour before meals and shall be stored at a cool and sealed place.

The present invention targets on the clinical efficacy of the different pathological forms of ALS disease manifestation. First, three herbal formulas, An Shen, Jie Fu Ling, and Yuan Qi Tangs are identified and combined to address the phenotypes of limb and bulbar onsets successfully. Second, Xiao Chai Hu Tang (Bupleurum chinense) is designed to take advantage of its functions of homeostasis, anti-side-effects, and anti-toxicities. Third, Riluzole is added as glutamate inhibitor to enhance neuron protection. All these implementations are established in an internal Clinical Trial with 214 patients to demonstrate its potential for the future therapy of ALS, as further illustrated in the paragraphs below.

### Example

The present invention is further supplemented with an internal human clinical trial using the Chinese herbals and Riluzole. The trial is based on the observation of ALS patients who received Chinese herbal treatments during a period of 10 years. The internal clinical trial, without Institutional Review Board (IRB) or ALSFRS-R, external review or follow ups, of ALS patients, are initiated with digital data recording between February 2004 and August 2014.

There are 500 registered patients from February 2004 to August 2014. But the records of 15 youth and babies are removed since the children motor neuron disease is not known as ALS. Among the remaining, about one-third had electromyography. 271 patients have records where the effectiveness of the decoction cannot be identified unambiguously. One patient has died a week after entering the program. As 286 are disqualified for lack of complete personal data, only 214 is remained as active participants.

The remaining 214 patients have relatively complete clinical data using TCM, and by serendipity, the ALS drug Riluzole (only one approved by FDA at the time) is used by significantly numbers of patients (14 patients use it concurrently with TCM and 19 patients use it sequentially and separately).

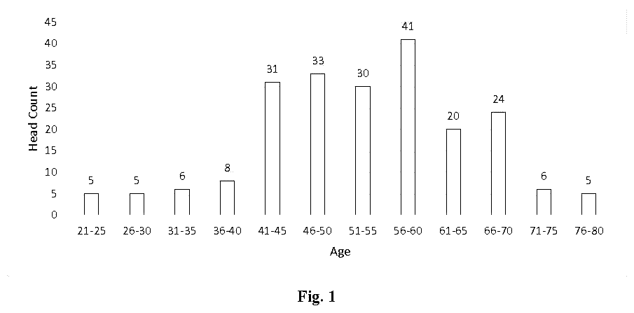

The majority 147 (70%) patients were from outside of Shanxi Province, 3 patients from the United States and one patient from Hong Kong. In most cases, the patient visits the clinic but left for home after the first diagnostic examination in which the patient also receives the prescription and dispensing of decoctions. Most patients are not to be seen by the physicians again. Because a large number of the patients come from the remote farming regions or small cities all over central and northern China where the public transportation was not well developed, returning visits for these handicapped patients to visit the doctors were not possible. The remaining contacts with the Clinic are by phone or email. The patient does not know the doctors and the doctors, with only exceptional cases, hardly knew any of the 214 patients after one interview. For practical purposes, the test is double-blind and randomized. No patient has advantages over others and there is no placebo group. Riluzole users have formed a small control unit. The age distribution is shown in FIG. 1 with a man-to-woman ratio of 1.78, close to that of Taiwan (1.65), China (1.7) and Hong Kong (1.72). FIG. 1 shows the age distribution of patients amounting to 214 total of head counts from the age 21 to 80 years old.

It may be further noted that most patients have gone through the major hospitals, but only ⅓ have used EMG. Second opinion can include hospitalized for observation. In these Chinese hospitals, ALS FRS, and ALS FRS_R are used exactly like the international standards. These patients' records remain in the hospitals and unavailable, but their final decisions came with referrals.

The three formulas, An Shen Tang, Yuan Qi Tang and Jie Fu Ling decoctions, all with Xiao Chai Hu, are formulated and are produced in a laboratory of the Research Clinic in Taiyuan, North China. An Shen Tang is used for limb-onset and Yuan Qi Tang for bulbar onset while Jie Fu Ling along with Xiao Chai Hu is taken by both groups. During the 10-year clinical studies, the herbal production and patient medical treatment culture have gone through a transition in China. The source of wild herbs has been nearly exhausted and cultivated herbs became the available standard. As a result, the herbal decoction effectiveness has become suspected. In addition, modern western medical practice has become widespread in the cities of China. Most of the ALS patients have first sought treatment in the major hospitals in Beijing or Shanghai and many have taken Riluzole. They typically have a few months' delay before being referred to the Taiyuan Clinic. It is known that without exception, all who have taken Riluzole have found it ineffective. But Riluzole is the only ALS medicine available with the standard of 50 mg twice per day for 2 months to a year, another fortunate common practice in China. For these reasons, the patients are divided into 100 per group so that the time dependent response can be recognized in the data set. The summary of the ALS patients treated by Chinese herbal medicine and Riluzole which recorded by three clinicians of the Research Clinic in Taiyuan and are shown in Table 1.

The first 100 patients in Table 1 below have a slight edge in the “improved” column so the change of herbal source and delay by getting treatment by the major city hospitals after 2005 have an effect but not of major proportion. In fact, the statistics are very well-behaved including columns of unchanged, decline and deceased. It is particularly comforting that the change from wild natural herbs to cultivated herbs provides evidence that the herbal decoction can be counted on with the quality comparable to the natural wild plants and roots. The low “deceased” figure must be discounted because of poor reporting. The Chinese culture or tradition is unlikely to report the sad ending. Nevertheless, the large number of improved and unchanged should lead to a lower mortality rate.

TABLE 1

| Total Patients = 500 | | | | | | |
|---|---|---|---|---|---|---|
| 10 Sep. 2016 | | Clinical Record Study | | | | |
| Dates of first | | 214 Patients under treatment | | | Riluzole | |
| consultation | Disqualified | Improved | Unchanged | Decline | users | Deceased |
| 2004 Feb. 10 | 53 | 33 | 10 | 7 | 4 | 2 |
| 2005 Sep. 1 | | | | | | |
| 2005 Sep. 3 | 57 | 29 | 9 | 7 | 8 | 2 |
| 2006 Jun. 23 | | | | | | |
| 2006 Jul. 19 | 61 | 26 | 7 | 6 | 8 | 1 |
| 2008 Feb. 20 | | | | | | |
| 2008 Feb. 21 | 63 | 25 | 7 | 8 | 7 | 1 |
| 2010 Apr. 1 | | | | | | |
| 2010 Apr. 2 | 52 | 23 | 11 | 6 | 7 | 3 |
| 2014 Aug. 8 | | | | | | |
| Total | 286 | 136 | 44 | 34 | 33 | 9 |
| Percentage | | 63.6-0% | 20.6% | 15.9% | 15.4% | 4.2% |

An “improved” rating is based on the patient's report, e.g., turning better in using upper arms, hand, or leg, able to use chopsticks, choking less frequent or disappeared, tongue stiffness reduced, taking normal food again, swallowing better, less saliva, improved speech, better mental alertness, able to go to toilet etc. “Unchanged” indicates no improvement or deterioration; symptoms stayed the same. “Decline” means the decoction is not effective and symptom of progressive deterioration is evident.

In Chinese, the word is “turn better” for “improve”. This condition is spoken or written by the patient and recorded by the attending physician. The patient has lost some functions such as speaking clearly, swallowing, drinking without chocking, using chopsticks, writing, walking, moving up the stairs, no dripping saliva, appetite, high spirit, mental alertness, cleaning, dressing, etc. After taking the herbal medicine, some of these specific functions have returned. For a typical ALS patient using Riluzole, the deterioration continued and no improvement or turning around was observed. This is why the standards of “improved” “unchanged” and “decline”, which are very different from the standards use for ALS FRS, are set.

Further, Table 2 below presents the results of the separation of limb and bulbar onsets with the prescribed medication.

TABLE 2
ALS patients treated with TCM (Inclusive and Exclusive of Riluzole users)

| Body Part | | | | | |
|---|---|---|---|---|---|
| (Number of | | | | | |
| Patient) | Medication | Improved | Unchanged | Declined | Deceased |
| Limb onset | An Shen Tang | 41 (46.6%) | 28 (31.8%) | 18 (20.5%) | 1 (1.1%) |
| (88) | Ji Fu Ling | | | | |
| Bulbar onset | Yuan Qi Tang | 79 (65.3%) | 24 (19.8%) | 10 (8.3%) | 8 (6.6%) |
| (121) | Ji Fu Ling | | | | |
| Limb onset | An Shen Tang | 6 (60.0%) | 3 (30.0%) | 1 (10.0%) | 0 |
| (10) | Ji Fu Ling | | | | |
| | Riluzole | | | | |
| Bulbar onset | Yuan Qi Tang | 18 (78.3%) | 5 (21.7%) | 0 | 0 |
| (23) | Ji Fu Ling | | | | |
| | Riluzole | | | | |
| Limb onset | An Shen Tang | 35 (44.9%) | 25 (32.0%) | 17 (21.8%) | 1 (1.3%) |
| (78) | Ji Fu Ling | | | | |
| | (No Riluzole | | | | |
| | users) | | | | |
| Bulbar onset | Yuan Qi Tang | 61 (62.2%) | 19 (19.4%) | 10 (10.2%) | 8 (8.2%) |
| (98) | Ji Fu Ling | | | | |
| | (No Riluzole | | | | |
| | users) | | | | |

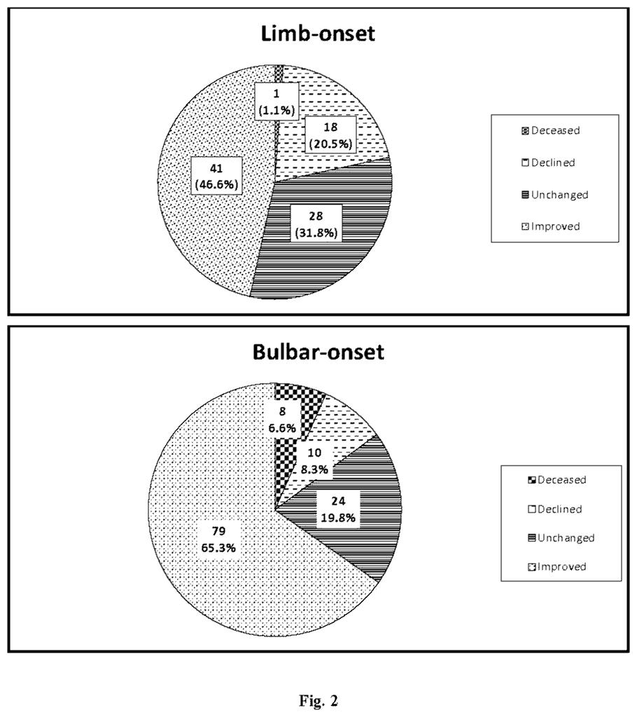

FIG. 2 depicts the effectiveness on the limb and bulbar onsets along with prescribed medication in percentage in the present invention. The end point is designated as “improved”, ‘unchanged” “decline” and “deceased” based on original admission interview and subsequent phone calls to three attending physicians. Face-to-face follow-up interviews are prohibitive because of the large distance of underdeveloped countryside and lack of public transportation. Phone calls provide the only link to check the patients' conditions and drug refills. Having Riluzole alone has no observable effect. FIG. 2 shows that with TCM alone, of 214 remained, 135 (63%) are found to have improved, regaining some lost functions, 42 (20%) has keep the same conditions having no improvement or deterioration, and 32 (15%) have declined. Among the treated limb-onsets, 41 (46%) out of 88 are found to have improved and the corresponding figures for bulbar onset are 83 (68%) out of 121. Whilst 9 patients have died within the period of treatment.

By serendipity, Riluzole users form a natural control group. Riluzole has been found as ineffective in China (between 2004-2015). However, since it is the only FDA approved drug for ALS, all hospitals have Riluzole available and most distributed as free gifts by the manufacturer. The Clinic's physicians are practicing herbal decoctions and the use of Riluzole is discouraged as useless but not forbidden. Among the 209 patients, 33 patients have used Riluzole. 19 patients take it prior to using the herbal decoction and 14 patients use the drug concurrently with the herbal medicine. These represent the control group.

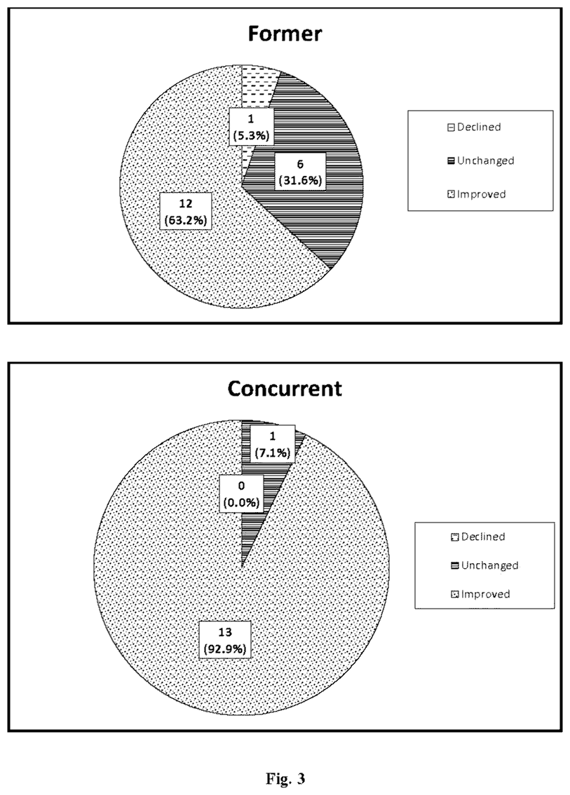

FIG. 3 illustrates the data from the control group, comparing patients treated with Riluzole alone to those receiving Riluzole in conjunction with Chinese herbal medicine. There are 14 patients use both TCM and Riluzole concurrently, and the result shows that 13 out of 14 have improved (92.8%) and 1 patient unchanged, without declined or death. Given to the patients by the drug maker free, Riluzole is taken by the patients at their own free-will. In these data, it was unambiguous that taken both TCM and Riluzole at the same time provide the best therapy. FIGS. 2 and 3 are prepared in 2016.

According to the Treatise of Cold Damages, which is also known as Shang Han Lun, the human body is a tripartite compartmental system: (1) Surface or outer layer: parts exposed to air including skins, nerves, respiratory and sensory systems, lung being the principal organ; (2) Inner layer: parts exposed to food intake and processing including digestive system, liver, kidney, pancreas and intestines, the stomach is the principal organ. (3) Half-in and half-out or center layer: any part not included in (1) and (2) above. The heart is its principal organ. The brain is a part of the center layer but counted as subordinate to the heart.

The justification appears connected with the pathogens since air and foods are the carrier agents of diseases, particularly in ancient times when internal source of diseases, e.g., genetic mutation, was not known or not counted. But of more interest is the rationale of various herbal prescriptions. Shang Han Lun makes use of the tripartite division to establish the principal herbal formula for each sub-system. Since ALS is a manifestation of neural and muscular diseases, it belongs to the central compartment. A typical recipe will require the deployment of the central principal formula (Minor Bupleurum Decoction or XCHT) or its variations. If there are two principal formulas, the present invention uses both and their combination.

From the clinical data, the decoction for coordination and regulation of whole body in Chinese medicine appears as functional and may even be effective or good enough; but the isolation of disease parts would need further improvements. In other words, the defensive element of the herbal recipe is nearly perfect, but the offensive is full of loopholes. Even though there is an overall improvement of 46% for limb-onsets and 68% for bulbar onsets, some more improvement is desirable. A clinical trial is needed to validate the data of clinical administration. The focus on patients with bulbar onset symptoms is suggested. Bulbar symptoms belong to the final stage of the disease. Many of the patients on the 485 patients have started with limb-onset but are progressed to the bulbar stage. Therefore, all serious patients in the records have been treated by Yuan Qi and Jie Fu Ling Tang. The Taiyuan Clinic has used the decoctions for tens of thousands of patients. The basic principle of treatment follows the simple rule that if in doubt clinical performance is the only meaningful judgement. This was how the current formulas arrived at. The recipes are ready for clinical tests to measure their efficacy since the toxicity is no longer in question.

In regard to the question of localization and isolation of the disease, it appears that Chinese herbal medicine is ideal to provide the necessary regulation and conditioning to arrive at tissue homeostasis for ALS. In the example, Jie Fu Ling is the modified decoction to provide the background for the functions of coordination and harmonization. By adding Yuan Qi Tang, the function of isolation and localization appears to have taken place. To find out the real pharmaceutical healing functions, the level of cell and molecular biology are to be explored further. But in this part the Chinese herbal medicine is woefully inadequate. With a composite of many herbs, there must be hundreds of chemicals involved and using the analytical chemical method of study may not be the most effective way. Thus, the present invention brings the ancient Chinese method into the modern world and integrate it with the latest molecular know how.

In reading the patient records more than a dozen times to make sure the numerical data are self-consistent; a repeated statement that all users of Riluzole agreed that the drug is completely ineffective or just useless are found. Nevertheless, a few patients have insisted keep using it while the Chinese herbal decoction is administered. Since Riluzole is FDA approved, it indicates some positive molecular functions have been achieved. The reasoning led to the examining the records of patients who have taken the Riluzole before or concurrently with the Chinese herbal decoction. There are 33 Riluzole users, or 15.8% of the 209 patients. These are separated into 19 prior users and 14 concurrent users. The results are shown in Table 3 below. The former group refers to those patients who have previously received Riluzole treatment, followed by administration of herbal decoction (i.e., Riluzole treatment was discontinued), whilst the concurrent group refers to individuals who have been simultaneously receiving both Riluzole and herbal decoction treatments.

TABLE 3
Patients taken Riluzole and Herbal Decoction

| Riluzole Users | Improved | Unchanged | Declined |
|---|---|---|---|
| All (33) | 25 | 7 | 1 |
| | 75.8% | 21.2% | 3.0% |
| Former (19) | 12 | 6 | 1 |
| | 63.1% | 31.6% | 5.3% |
| Concurrent (14) | 13 | 1 | 0 |
| | 92.9% | 7.1% | 0% |

The data for the prior users indicate their effective grouping in improvement is slightly off from the typical herbal user, 63% vs 64.6%, but within the standard deviation. However, the concurrent users are 13 (93%) out of 14 counted as improved, only one is considered unchanged, not declined. Thus, the addition of Riluzole has significantly improved the decoction data as a group. Yet having the pure function of chemical treatment without herbal regulating and harmonizing, Riluzole is ineffective by itself. While the Chinese decoctions have both defensive and offensive elements, they lack precision in isolation or attacking function. Riluzole appeared to have made the Chinese decoction more effective. The Riluzole patients as a group having both the ineffective label (prior users) and excellent healer (concurrent users), can be considered as a randomized control unit to highlight the actual improvement when the Chinese herbal concoction is incorporated as the foundational treatment. With the proper drug for the background, the idea of trying other drugs may conceivably produce positive gain for future patient treatment. Nevertheless, from the above description, it is evident that a cure of human for Amyotrophic Lateral Sclerosis has been demonstrated.

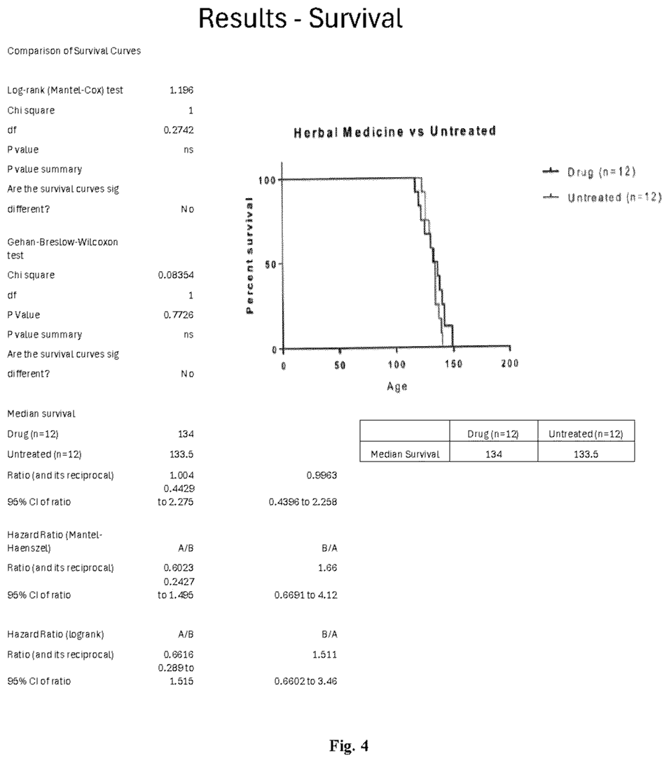

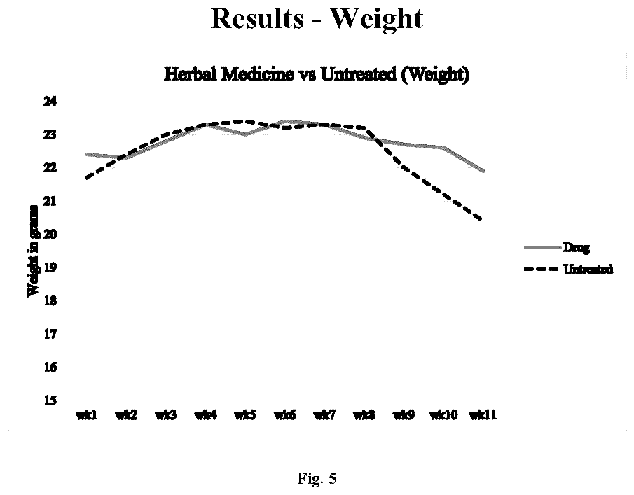

For the animal trial, which is conducted in 2017, the animals used are SOD1-G93A transgenic ALS mice known genetically as familial fALS (SOD1). The TCM, without Riluzole, is designed in China for Chinese, for sporadic sALS, is a mismatch. The only meaningful data is that no toxicity is observed. However, as shown in FIGS. 4 and 5 on weight and survival of mice in 11 weeks, it is important to note the result shows the drug safety for TCM treatment without Riluzole. FIG. 4 depicts the survival outcomes observed in the animal trial, comparing the use of herbal medicine with the untreated control group, and FIG. 5 illustrates the weight fluctuations observed in 24 SOD1 transgenic mice over an 11-week period, comparing those administered with Chinese herbal medicines without Riluzole to those left untreated.

In Chinese medicine, herbal recipe represents a dozen or more ingredients and hundreds of chemicals. In contrast with western drugs, it is not a single chemical element or compound. In fact, since they are herbals, they are the leaves, flowers, fruits, barks, and roots that have gone through cycling and recycling through many evolutionary eons as plants. In addition, medical herbs in China have also gone through human selection for more than 5000 years. If one looks at these elements as clinical trials components, for safety record, they are in a different class compared to western drugs with targeted chemicals or compounds. The side effects and toxicities of the herbal components are known to be low, with special selected are negligible. In short, herbal components are suitable integrated partners for current or future ALS medications. This present invention shows that for the first time ALS is treatable and patients have been found to have recovered from the dreadful disease as reported.

The present invention explained above is not limited to the aforementioned embodiment and drawings, and it will be obvious to those having an ordinary skill in the art of the prevent invention that various replacements, deformations, and changes may be made without departing from the scope of the invention.

1. A method for diminishing effects of Amyotrophic Lateral Sclerosis (ALS) or neurodegeneration in a predisposed subject comprising administering to the predisposed subject effective amounts of Traditional Chinese Medicine (TCM) ingredients selected from a group consisting of:
(1) Ge Gen (Radix puerariae, Lobed Kudzuvine Root), (2) Dang Gui (Radix angelicae, Chinese Angelica or Female Ginseng), (3) Dan Shen (Salviae miltiorrhizae, Red sage), (4) Dang Shen (Codonopsis radix, Asiabell Root), (5) Huang Qi (Radix astragali, Milkvetch Root), (6) Zi Su Zi (Perillae fructus, Purple Perilla Fruit), (7) Da Zao (Jujubae fructus, Jujube), (8) Chai Hu (Bupleuri radix, Chinese Thorawax Root) (9) Huang Qin (Scutellariae radix, Baikal Skullcap Root), (10) Hong Hua (Carthami flos, Safflower), (11) Yu Jin (Curcumae radix, Turmeric), (12) Da Huang (Rhei radix, Rhubarb), (13) Hua Jiao (Zanthoxyli pericarpium, Pricklyath Pericarp), (14) Gan Cao (Glycyrrhizae radix, Liquorice), (15) Mai Dong (Ophiopogonis radix, Dwarf Lilyturf Root), (16) Wu Wei 2i (Schisandrae chinensis fructus, Chinese Magnoliavine Fruit), (17) Fu Zi (Aconiti lateralis, Monkshood Daughter Root), (18) Ren Shen (Ginseng radix, Ginseng), (19) Fu Ling (Poria cocos, Buead Tuckahoe,) (20) Shi Gao (Gypsum fibrosum, Gypsum), (21) Mu Li (Ostreae concha, Oyster shell), (22) Gui Zhi (Cinnamomi ramulus, Cassiabarktree Twig), and (23) Che Qian Zi (Plantaginis semen, Asiatic Plantain Seed), or mixtures thereof; or,
extracts thereof in amounts equivalent to the amounts of the raw materials of the group of ingredients.

2. The method according to claim 1, wherein the effect of neurodegeneration in the predisposed subject is due to Amyotrophic Lateral Sclerosis (ALS).

3. The method according to claim 1, wherein the TCM is in a form of decoctions, granules, capsules, powders, water pills, tablets, mixtures, oral liquids or other dosage forms.

4. The method according to claim 1, wherein the TCM is administered to the predisposed subject for a period of at least 3 months consecutively for effective results.

5. The method according to claim 1, wherein the TCM is administered on its own or in a combination with an agent used in Western medicine with a glutamate inhibitor, including Riluzole, Edaravone, or Toferson.

6. The method according to claim 5, wherein the agent used in the Western medicine is provided for simultaneous, separate or sequential use in treatment of the predisposed subject.

7. The use of the traditional Chinese medicine (TCM) components to manufacture the medicament of the treatment for the neurodegenerative disease according to claim 3 including but not limited to repairing cell damages and muscle healing.

8. A product comprising a Chinese medicament comprising of 10-15 units of Huang Qi (Radix astragali, Milkvetch Root), 5-10 units of Ge Gen (Radix puerariae, Lobed Kudzuvine Root), 3-6 units of Dang Gui (Radix angelicae, Chinese Angelica or Female Ginseng), 1.5-3 units of Hong Hua (Carthami flos, Safflower), 3-6 units of Dan Shen (Salviae miltiorrhizae, Red Sage), 1.5-3 units of Yu Jin (Curcumae radix, Turmeric), 1-2 units of Da Huang (Rhei radix, Rhubarb), 1.5-3 units of Chai Hu (Bupleuri radix, Chinese Thorawax Root), 1.5-3 units of Huang Qin (Scutellariae radix, Baikal Skullcap Root), 3-6 units of Zi Su Zi (Perillae fructus, Purple Perilla Fruit), 3-6 units of Dang Shen (Codonopsis radix, Asiabell Root), 1-2 units of Huajiao (Zanthoxyli pericarpium, Pricklyash Pericarp), 1-2 units of Gan Cao (Glycyrrhizae radix, Licorice), and, 1.5-3 units of Da Zao (Jujubae fructus, Jujube).

9. A product comprising a Chinese medicament comprising of 1-2 units of Ren Shen (Ginseng radix, Ginseng), 1.5-3 units of Fu Zi (Aconiti lateralis, Monkshood Daughter Root), 1.5-3 units of Fu Ling (Poria cocos, Bread Tuckahoe), 3-6 units of Mai Dong (Ophiopogonis radix, Dwarf Lilyturf Root), 1.5-3 units of Wu Wei Zi (Schisandrae chinensis Fructus, Chinese Magnoliavine Fruit), and, 6-10 units of Huang Qi (Radix astragali, Milkvetch Root).

10. A product comprising a Chinese medicament comprising of 3-6 units of Shi Gao (Gypsum fibrosum, Gypsum), 1-2 units of Gui Zhi (Cinnamomi ramulus, Cassiabarktree Twig), 3-6 units of Mu Li (Ostreae concha, Oyster Shell), 1-2 units of Che Qian Zi (Plantaginis semen, Asiatic Plantain Seed), 1.5-3 units of Chai Hu (Bupleuri radix, Chinese Thorawax Root), 1.5-3 units of Huang Qin (Scutellariae radix, Baikal Skullcap Root), 3-6 units of Zi Su Zi (Perillae fructus, Purple Perilla Fruit), 3-6 units of Dang Shen (Codonopsis radix, Asiabell Root), 1-2 units of Hua Jiao (Zanthoxyli pericarpium, Pricklyash Pericarp), 1-2 units of Gan Cao (Glycyrrhizae radix, Licorice), and, 1.5-3 units of Da Zao (Jujubae fructus, Jujube).
